(19) United States
(12) Patent Application Publication
(10) Pub. No.: US 2007/0282313 A1
(43) Pub. Date: Dec. 6, 2007

(54) METHOD AND APPARATUS TO GUIDE LASER CORNEAL SURGERY WITH OPTICAL MEASUREMENT

(75) Inventors: David Huang, South Pasadena, CA (US); Jonathan C. Song, South Pasadena, CA (US); Yan Li, Pasadena, CA (US); Maolong Tang, San Gabriel, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 11/757,195

(22) Filed: Jun. 1, 2007

Related U.S. Application Data
(60) Provisional application No. 60/810,542, filed on Jun. 1, 2006.

(51) Int. Cl. A61B 18/18

(52) U.S. Cl. 606/5

(57) ABSTRACT

Optical coherence tomography (OCT) is used to map the surface elevation and thickness of the cornea. The OCT maps are used to plan laser procedures for the treatment of an irregular, opacified or weakened cornea, and in the treatment of refractive errors. In the excimer laser phototherapeutic keratectomy (PTK) procedure, the OCT data is used to plan a map of ablation depth needed to restore a smooth optical surface. In the excimer laser photorefractive keratectomy procedure, OCT mapping of epithelial thickness is used to achieve clean laser epithelial removal. In femtosecond laser anterior keratoplasty procedure, OCT data is used to plan the depth of femtosecond laser dissection to remove an anterior layer of the cornea, leaving a smooth recipient bed of uniform thickness to receive a disk of donated corneal tissue. The linkage of an OCT system to a precise laser surgical system enables the performance of new procedures that are safer, less invasive and produce faster visual recovery than conventional surgical procedures.

### CROSS-REFERENCE TO RELATED APPLICATIONS

This application claims an invention which was disclosed in Provisional Application No. 60/810,542 filed Jun. 1, 2006 entitled “METHOD AND APPARATUS TO GUIDE LASER CORNEAL SURGERY WITH OPTICAL MEASUREMENT”. The benefit under 35 USC §119(e) of the U.S. provisional application 5 is hereby claimed. The above priority applications are hereby incorporated herein by reference.

### STATEMENT REGARDING FEDERALLY SPONSORED RESEARCH AND DEVELOPMENT

Not applicable.

### FIELD OF THE INVENTION

The invention pertains to the field of opthalmology. More particularly, the invention pertains to methods for guided corneal surgery and apparatuses for performing thereof.

### BACKGROUND OF THE INVENTION

The cornea is the transparent front part of the eye that covers the iris, pupil, and anterior chamber, providing most of an eye's optical power. Together with the lens, the cornea refracts light, and as a result helps the eye to focus, accounting for approximately 75% of its focusing power, compared to 25% from the lens. The cornea contributes more to the total refraction than the lens does, but, whereas the curvature of the lens can be adjusted to “tune” the focus depending upon the object's distance, the curvature of the cornea is fixed. FIG. 1 shows a schematic diagram of the human eye.

The cornea has unmyelinated nerve endings sensitive to touch, temperature and chemicals; a touch of the cornea causes an involuntary reflex to close the eyelid. Because transparency is of prime importance the cornea does not have blood vessels; it receives nutrients via diffusion from the tear fluid at the outside and the aqueous humour at the inside and also from neurotrophins supplied by nerve fibres that innervate it. In humans, the cornea has a diameter of about 11.5-12.5 mm and a thickness of 0.5 mm-0.6 mm in the center and 0.6 mm-0.8 mm at the periphery. Transparency, avascularity, and immunologic privilege makes the cornea a very special tissue.

In humans, the refractive power of the cornea is approximately 43-46 dioptres, roughly three-quarters of the eye's total refractive power. Several major eye disorders are related to the impairment of cornea's refractive power, among which are myopia (near-sightedness or excess focusing power), hyperopia (farsightedness or shortage of focusing power), astigmatism (uneven focusing power), scars (opacification of the cornea), and keratoconus (thinning and protrusion of the cornea). Surgical treatments for improving and/or restoring the refractive state of the cornea are collectively called refractive eye surgery.

The most common methods of laser corneal surgery today use excimer lasers to reshape the curvature of the cornea. Successful refractive eye surgery can help to reduce common vision disorders such as myopia, hyperopia and astigmatism. According to surveys of members of the American Society of Cataract and Refractive Surgery, approximately 948,266 refractive surgery procedures were performed in the United States during 2004 and 928,737 in 2005.

Although laser corneal surgery are now routine, current methods still have certain limitations that remain unaddressed, particularly in situations where a subject's eyes have highly irregular cornea geometry or have become opaque.

For example, in the commonly practiced refractive surgery procedures known as phototherapeutic keratectomy (PTK) and transepithelial PTK, excimer lasers are used to remove minor surface irregularities and anterior stromal opacities. In the usual PTK technique, the corneal epithelium in the area to be ablated is scraped off with a blade and any mechanically separable nodular scar is removed with blade and forceps. The remaining rough corneal surface is masked with a moderately viscous fluid which covers up the depressions while leaving the peaks exposed for selective laser ablation. In transepithelial PTK, the ability of epithelial cells to grow back in varying thickness is utilized. Fluid masking agent and transepithelial ablation are effective in removing small scale irregularity and produce a smoother surface. But these techniques still leave medium to large scale irregularities of the corneal surface untreated.

In another example, Placido-ring based corneal topography systems project illuminated concentric rings on the front surface of the cornea. The reflected images are captured on a digital video camera. The curvature of the cornea is measured from the ring spacing on the image. An elevation map could then be computed using an integration algorithm. The elevation map could be used to guide laser ablation and correct corneal surface irregularity with good results. However, Placido-ring topography can capture data only when there is a relatively smooth surface and good tear film stability. Most eyes with visually significant corneal scars cannot get a valid topography reading due to excessive surface irregularity or unstable tear film. Thus topography-guided PTK has only limited applicability and cannot help the patients with more severe corneal problems.

Currently, corneas that are too irregular, scarred or distorted to be corrected by spectacles, contact lens and PTK are usually treated by corneal transplantation. Corneal transplantation is one of the most commonly performed organ transplantation surgeries. According to the Eye Bank Association of America, 33,260 corneal transplantation procedures were performed in the year 2000. Although the medium term success rate of corneal transplantation for adult patients is good (>90%), it is poor to fair (48-74%) in infants and children. Graft survival in the very long term may be poor.

Most corneal transplant surgeries today involve the full thickness of the cornea in a procedure called “penetrating keratoplasty” or PK. A rejection reaction can occur to any layer of the cornea, but most graft failures result from rejection of the corneal endothelium because it has no regenerative capability and its function is critical to corneal transparency and clarity. The endothelium is a thin layer of cells that actively pumps water out of the cornea to maintain its clarity. Loss of endothelial density beyond a critical value causes swelling and opacification of the cornea. Even without a rejection reaction, transplanted endothelium degenerates at an accelerated rate over at least 2 decades. The endothelial loss after PK fits well with a biexponenential model and the slow component of decay has a half-life of 21 years after PK in a long-term study. Although there is no study that tracks transplants beyond 20 years, extrapolation indicates that most transplants would fail after 3 decades.

Although a failed corneal transplant can be replaced with a new graft, the success rate of repeat surgery is lower (46-68%). With graft rejection, there are often changes in the host eye such as abnormal growth of blood vessels into the cornea, adhesion between the iris and the cornea and elevated intraocular pressure. These inflammation-related changes increase the risk of repeat graft rejection and accelerated graft degeneration.

Given the long term risks of PK, it is best avoided if other alternatives are possible.

If the corneal pathology does not involve the endothelium, a partial thickness transplantation of the anterior layer of the cornea (also called lamellar keratoplasty or LK) could potentially offer a way to avoid the endothelial problems associated with PK. However, surgeons usually do not choose to perform LK today because the procedure is technically difficult, carrying the risk of penetrating into the eye in uncontrolled fashion or leaving an irregular match between the corneal layers resulting in poor quality of vision. The visual acuity achieved in LK is inferior to PK by approximately one line on the Snellen chart according to a recent review of the literature. The primary limitation of LK is the uneven dissection and matching of the donor tissue (graft) and the recipient bed of the host cornea. Good optical outcome requires that both lamellar surfaces be smooth, and the thickness and diameter be regular and matched.

Several techniques have been developed for LK. Manual dissection of the host stromal bed was the first to be tried and its main draw back is the unevenness of the dissection. Since there is less interlamellar connection in deeper corneal stroma, some investigators advocated very deep dissection or even dissection down to the Descemet's membrane. The chief limitations of this approach are the difficulty of the dissection and the increased risk of uncontrolled penetration into the anterior chamber. The microkeratome, an automated device incorporating an applanation surface and oscillating blade to cut the cornea at a preset depth, has been used to improve the smoothness and ease of lamellar incision. The shortcoming of the microkeratome is the resulting variations in diameter, shape, and depth of the lamellar disk. The excimer laser has also been used to prepare the host stromal bed. However, the technique used so far does not remove the uneven contour in the host cornea.

In a more advanced form of corneal surgery, femtosecond lasers are used. In such procedures, concentrated energy in extremely short pulses that are typically several tens or hundreds of femtoseconds (one million billionth of a second) in duration are directed at the cornea of a subject. A pulse that short creates a microscopic explosion when focused inside the cornea. Millions of femtosecond pulses, when properly controlled, create an extremely precise cut inside the cornea. Intralase, Inc. has developed a commercially successful infrared femtosecond laser to assist in the flap dissection portion of the LASIK procedure. The laser has also been recently used to dissect the recipient cornea in penetrating keratoplasty and deep lamellar endothelial keratoplasty. But applications in anterior lamellar keratoplasty are yet to be demonstrated.

Femtosecond laser dissection of the cornea is usually performed in a plane of constant depth from the corneal surface. For LK on an irregular cornea, this would produce an irregular recipient bed as shown in FIG. 2.

The femtosecond laser is controlled by computers and can be made to dissect the cornea with widely varied patterns. However, in order to apply femtosecond laser, optimally customized cuts at high precisions are required. Without a method to precisely measure corneal depth, applications of femtosecond laser are not feasible.

Several methods are currently available for measuring corneal depth. The widely known slit-scanning systems (Orbscan II by Bausch & Lomb, Rochester, N.Y. and Pentacam by Oculus Gmbh, Germany) can obtain reproducible corneal thickness maps in normal eyes. However, slit-scanning systems tend to underestimate corneal thickness when there is subepithelial haze or stromal opacity. This is due to the limited axial resolution of slit-scanning technology.

Ultrahigh frequency ultrasound imaging (Artemis by Ultralink, Inc.) can also map corneal thickness. However, it requires immersing the eye in a fluid bath because ultrasound cannot pass through air. The inconvenience and discomfort associated with the fluid bath makes it unsuitable for clinical applications.

Placido-ring based corneal topography systems work well on corneas with a smooth surface. However, the quality of topography data depends on the specular reflection from a smooth tear film and it cannot capture the surface of corneas with severe irregularity or unstable tear film. Therefore Placido-ring topography-guided laser ablation cannot be applied to highly irregular corneas that need the treatment most.

Wavefront-guided laser treatment works well in reducing the aberration of the eye in normal eyes. However, the wavefront sensor cannot obtain a valid measurement on highly aberrated eyes, eyes with extreme refractive error, eyes with corneal opacity or cataract, eyes with unstable tear film and many eyes with intraocular lens implants. In our experience, the wavefront sensor cannot obtain a valid measurement in the great majority of patients with visually significant corneal scar or irregularity. Therefore wavefront-guided laser ablation cannot help most eyes with significant corneal irregularity.

Despite the variety of techniques currently available for measuring cornea thickness, none of the currently available prior art methods can meet the requirement of precision, flexibility, and ease-of-use needed to realize routine customized corneal surgeries.

### SUMMARY OF THE INVENTION

In view of the above, it is one object of the present invention to provide a method for performing routine customized corneal surgeries. Successful performance of such high-precision operations will largely hinge on the precision and flexibility of the tools available. Therefore, in one aspect, the present invention also provides systems and computerized tools for achieving the desired manipulation.

In a first aspect, the present invention provides a system for performing corneal surgery, comprising; corneal mapping device for mapping a cornea tomograph to a predetermined precision; and an ablative laser linked to the optical coherence tomography device, wherein actions of the ablative laser are guided by a treatment plan based on the cornea tomograph obtained by the corneal mapping device.

In a second aspect, the present invention also provides a method for performing laser phototherapeutic keratectomy, comprising the steps of: (a) obtaining a tomograph of the cornea; (b) generating a map of the cornea based on the tomograph, wherein the map contains information of both thickness and anterior elevation of the cornea at a precision of at least 2 microns; (c) computing a treatment plan based on the cornea thickness map, wherein the treatment plan comprises ablation patterns to be performed by a laser; and (d) ablating the cornea with a ablative laser according to the ablation pattern of the treatment plan.

In a third aspect, the present invention also provides a method for performing femtosecond laser anterior keratoplasty, comprising the steps of: (a) obtaining a tomograph of the cornea of a subject with optical coherence tomography; (b) converting the optical coherence tomograph into a map of corneal thickness; (c) designing a laser dissection treatment plan base on the corneal thickness map; (d) performing intrastromal dissection according to the treatment plan using a femtosecond laser; (e) removing dissected anterior corneal tissues to leave a recipient bed; and (f) replacing the removed tissues with a disk of donated corneal tissue.

In a fourth aspect, the present invention further provides a computer configured such that it is capable of automating and controlling a corneal mapping device in concert with an ablative laser to perform the methods of the present invention.

In a fifth aspect, the present invention also provides computer readable medium having encoded thereon computer software that implements the methods of the present invention.

Major advantages for linking an OCT system to a precise laser surgical system in accordance with the present invention enables the performance of new procedures that are safer, less invasive and produce faster visual recovery than conventional surgical procedures.

Other aspects and advantages of the invention will be apparent from the following description and the appended claims.

### BRIEF DESCRIPTION OF THE DRAWINGS

FIG. 1 shows a schematic diagram of the eye.

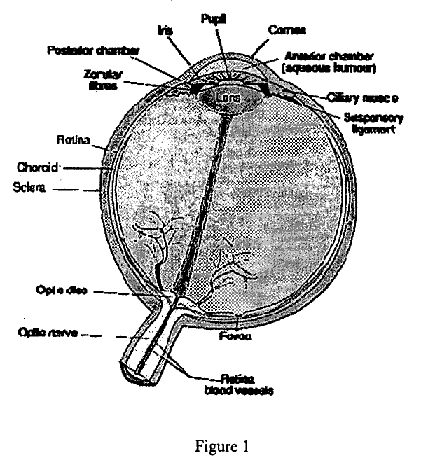

FIG. 2 shows an exemplary cross sectional view of an irregular corneal bed.

FIG. 3 shows an exemplary system according to one embodiment of the present invention.

FIG. 4 shows an OCT scan of the cornea using a spoke pattern of radial lines (left). The scan data is to be processed to generate a map of corneal thickness (right). The map is divided into zones (red partition lines on right) and the average and minimum thickness is computed in each zone.

FIG. 5 shows the result of applying image processing steps according to one embodiment of the present invention to a meridional cross-sectional OCT image that consists of 128 axial scans across 10 mm acquired at 2 kHz. The axial resolution was 17 microns full-width-half-maximum (FWHM). (A) Unprocessed OCT with reflected signal amplitude represented on a logarithmic gray-scale. (B) OCT image after dewarping to correct for refraction at the air-corneal boundary. (C) An axial scan waveform is taken from the pericentral cornea. Reflected signal amplitude (log scale) is represented on the vertical axis while the axial (depth) dimension is represented on the horizontal axis. The arrows point to, from the left to right, the reflection peaks from the air-tear interface, the anterior stromal surface (Bowman's layer) and the posterior boundary (cornea-aqueous interface). (D) The anterior and posterior corneal boundary lines identified by the computer program are overlaid on the dewarped image.

FIG. 6 illustrates OCT-guided PTK, according to one embodiment of the present invention, for a scarred cornea with irregular surface. (A) OCT measures the irregularity of the anterior stromal surface (top line). An OCT maps is used to program excimer laser ablation that removes tissue from the anterior stroma and restore a smooth surface within the optical zone. The second line represents the surface after ablation. TZ=transition zone. (B) The epithelium and the tear film smooth over stromal irregularity by filling in the troughs and thinning over the peaks. Therefore the anterior corneal surface measured at the air-tear interface is different from that measured at anterior stroma surface. PTK can be performed after scraping off the epithelium to reveal the anterior stromal surface. Alternatively, transepithelial PTK starts the laser ablation from the pre-epithelial tear film.

FIG. 7 Left: An OCT image of a normal cornea obtained by the 830 nm FD-OCT (5 μm FWHM axial resolution). Right: An axial scan showed signal peaks (arrows from left to right) at air-tear, epithelium-Bowman's layer, Bowman-stroma, and stroma-aqueous interfaces.

FIG. 8 The OCT system scans the cornea using a spoke pattern of radial line (left). The scan data is automatically processed to generate a map of epithelium thickness (right).

FIG. 9 The epithelial ablation depth map (bottom) was calculated from the epithelium thickness map by the steps of extrapolation and low-pass filtering.

FIG. 10 shows a schematics diagram of the eye in (A) a natural state; and (B) with a contact plate applanating the cornea.

FIG. 11 shows (A) an incision edge design of a cornea in a flattened state; and (B) the same cornea in a relaxed native state.

FIG. 12 shows (A) an exemplary recipient cornea sectional image; and (B) the corresponding corneal thickness profile.

FIG. 13 shows (A) an exemplary design for an edge of the cornea graft recipient in a flattened state; and (B) the same cornea in its native state.

FIG. 14 shows another exemplary design for cutting the cornea of a cornea graft recipient.

FIG. 15 shows an exemplary donor cornea on top of a recipient cornea.

FIG. 16 shows the topography map of a keratoconic eye.

### DETAILED DESCRIPTION

The present invention will now be described in detail by referring to specific embodiments as illustrated in the accompanying figures.

As mentioned in the section above, one object of the present invention is to provide a method for treating eye disorders in patients whose cornea may have highly irregular geometry or may have become opaque. No known prior art method is capable of treating such eye defects. Accordingly, in a first aspect, the present invention provides a system for performing corneal surgery that is capable of delivering personalized treatment that is customized to take into account the variations among individual patients.

In general, a system according to embodiments of the present invention comprises a corneal mapping device for mapping a cornea tomograph to a predetermined precision, and an ablative laser linked to the mapping device. In such a system, the actions of the ablative laser are guided by a treatment plan based on the cornea tomograph obtained by the corneal mapping device.

The corneal mapping device may be any corneal mapping device so long as it is capable of obtaining and generating accurate maps of the cornea at a high speed. Cornea map, as used herein, refers to the collection of information that describes the geometric properties of the cornea in a coordinate reference frame. For instance, a cornea map may comprise of thickness data of the cornea, or surface elevation data, or a combination of both. The type of information contained in the map is not particularly limited so long as the data can be mapped onto a coordinate system representing the actual cornea. Preferably, optical coherence tomography (OCT) is used for this purpose and thickness data is collected to form a thickness map. OCT using the faster Fourier domain technology is preferred. Other suitable corneal mapping device may include high-frequency ultrasound or any other current or future corneal mapping device so long it meets the requirement of fast and high-precision.

Optical coherence tomography is an imaging technology that provides very detailed cross-sectional images (tomography) of internal tissue structure. Its principle is similar to RADAR and ultrasound imaging, where the instrument measures the round-trip delay time of reflected radio wave or ultrasound wave to determine the target structure in depth. Transverse scanning of the beam provides information about the lateral structure. In OCT, a beam of infrared light is directed at the sample and the delay of reflected light is measured. Because light travels extremely rapidly (30,000 kilometer/sec), it is impossible to directly measure the travel time of light with micron resolution. To overcome this limitation, the OCT system measures the delay of sample reflections indirectly, by its interference with a reference beam. The axial resolution of OCT is determined by the coherence length of the light source, hence the name “optical coherence tomography.” The resolution of OCT is very high, ranging from 2 to 20 micron full-width-half-maximum (FWHM). (Reference 1 gives a detail description of OCT, the entire content of which is incorporated herein).

In one embodiment, a high-speed OCT system that can obtain cross-sectional images of the cornea in a fraction of a second without touching the eye is used (see references 2-6, the entire contents of which are incorporated herein by reference). The corneal OCT system as used herein should have desirable characteristics in several respects:

1. Higher speed. Higher scanning speed are essential in order to obtain high resolution and minimize image distortion due to eye movement. Preferably, the scanning speed is from about 2 kHz to about 4 kHz (thousand axial scans per second), which can be accomplished using time-domain OCT technology. More preferably, the scanning speed is at least 20 kHz, which is commonly accomplished with Fourier-domain OCT technology. In yet another preferred embodiment, the scanning speed is at least 200 kHz, the maximum speed that has been demonstrated with Fourier-domain OCT.

2. Telecentric scanning. Preferably, the scanning optics is designed so the OCT beam is always parallel to the optical axis. This way, the resulting image is rectangular so that distortion of the image is minimized and accurate measurements are facilitated.

The ablative laser may be any laser suitable for eye surgery. Exemplary ablative lasers suitable for use in the present invention may include, but not limited to infrared lasers and ultraviolet lasers, Preferably, an ultraviolet excimer laser is used. In another preferred embodiment, a femtosecond infrared laser is used.

Femtosecond laser concentrates energy in extremely short pulses that are typically several tens or hundreds of femtoseconds (one million billionth of a second) in duration. A pulse that short creates a microscopic explosion when focused inside the cornea. Millions of femtosecond pulses, when properly controlled, create an extremely precise cut inside the cornea. Intralase, Inc. (Irvine, Calif.) has developed a commercially successful infrared femtosecond laser to assist in the flap dissection portion of the LASIK procedure. The laser has also been recently used to dissect the cornea of a subject in penetrating keratoplasty (see reference 7, the relevant portion of which is incorporated herein by reference) and deep lamellar endothelial keratoplasty (see reference 8, which is also incorporated herein by reference).

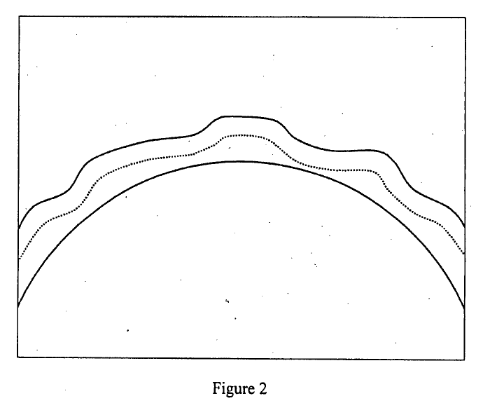

Femtosecond laser dissection of the cornea is usually performed in a plane of constant depth from the corneal surface. For LK on an irregular cornea, this would produce an irregular recipient bed (FIG. 2).

The femtosecond laser is controlled by computers and can be made to dissect the cornea with widely varied patterns. However, they have not been used to customize the lamellar cut at optimal depths because there has not been a precise method to measure the cornea.

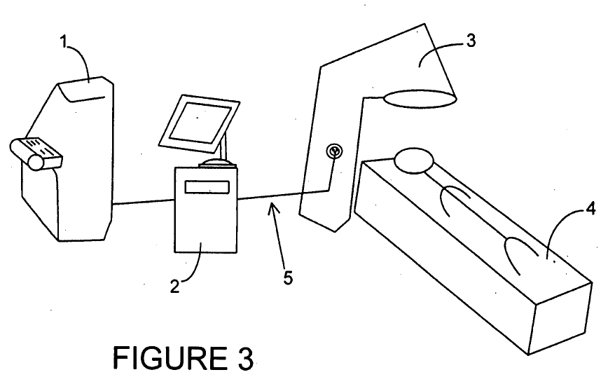

FIG. 3 shows a schematic diagram of an exemplary system of the present invention. Referring to FIG. 3, a corneal mapping device 1 such as a OCT device is linked to a femtosecond laser surgical unit 3 via a computer control unit 2. A subject 4 may first have his/her corneal map measured by the corneal mapping device 1. The measured map is then passed on to the computer 2 for treatment planning computation. The subject 4 is situated at the laser surgical unit 3 where the computer then controls the laser to perform the planned treatment.

Although the corneal mapping unit 1, the computer 2 and the femtosecond laser surgical unit 3 are shown in the figure separate units being linked together via the communication linkage 5, this is not necessary. The system may be modular, semi-integrated, or completely integrated to accommodate the specific needs of the operating environment. For example, in a large hospital setting, the separate units may be modularized and housed in separate geographic locations to accommodate and facilitate assembly line style operation. Because of the differences in time requirement, in a high volume, multi-function environment, the cornea mapping unit 1 may achieve better utilization as a separate module linked to the surgical unit. This way, when the surgical unit 3 is in operation, the corneal mapping unit 1 can continue to be utilized for other patients or other diagnostic uses.

On the other hand, for optimal registration between measurement and ablation, the corneal mapping unit 1 and the laser surgical unit 3 may form one integrated unit encased in the same housing. This way, there is no need for a separate mapping station and the entire surgical procedure from mapping to surgery may be done without having to move the patient around. One benefit is that the corneal mapping measurement and laser ablation coordinates are well matched due to being performed close in time and under the same eye fixation device. Furthermore, the same eye position measurement and tracking technique (for example, pupil tracking) can be used to establish the position of mapping and ablation on the cornea. An added benefit of this configuration is that the corneal mapping device 1 may continue to scan the eye of the patient even during surgical procedure so as to receive continuous updates of the corneal geometry throughout the operation.

The above mentioned configurations are only some possible examples. A skilled person in the art will appreciate that other configurations are also possible and the present invention is not particularly limited to any of the above mentioned limitations.

Having described the basic elements of a guided laser surgical system of the present invention, we now describe exemplary methods of the present invention.

In a second aspect, the present invention provides a method for performing laser phototherapeutic keratectomy, comprising the steps of: (a) obtaining a tomograph of the cornea; (b) generating a map of the cornea based on the tomograph, wherein the map comprise information of corneal thickness or anterior elevation of the cornea at a precision of at least 2 microns; (c) computing a treatment plan based on the corneal thickness map, wherein the treatment plan comprises ablation patterns to be performed by a laser; and (d) ablating the cornea with a ablative laser according to the ablation pattern of the treatment plan.

Keratectomy, as used herein, refers to the medical procedure of removing part of the cornea. In methods according to embodiments of the present invention, keratectomy is performed in a high-precision manner that draws on detailed information of the corneal geometry. Thus, in a first step, a high resolution tomograph of the cornea is obtained. This can be done, for example, with an optical coherence tomography device. In a second step, a detailed map of the cornea is generated from the tomography images. The map may be a thickness map, an anterior elevation map, or a combination thereof. In a following step, the map is then used to generate a treatment plan which will prescribe the precise dosing information for delivering the laser beam to the eye. The treatment plan is preferable in a computer or machine readable format that can be used to direct the actions of the ablative laser. Finally, the treatment plan is carried out by the ablative laser to perform the desired surgical cut.

The corneal map is preferably measured to a precision not less than 2 microns, more preferably not less than 1 micron. In one embodiment, the corneal map is a thickness map derived from OCT images and the treatment plan is based on the thickness information.

In prior art methods such as the slit-scanning systems, corneal thicknesses tend to underestimated when there is subepithelial haze or stromal opacity. The inventors of the present invention have discovered that by using an OCT system, more accurate measurement of corneal thickness may be achieved. In an experiment conducted by the inventors, 23 eyes of 19 patients with opaque cornea were imaged with a high-speed corneal OCT prototype (Carl Zeiss Meditec, Inc., Dublin, Calif.), slit-scanning tomography and ultrasound pachymetry. It was found that OCT produced results consistent with ultrasound measurements whereas slit-scanning tomography consistently underestimated corneal thickness in patients with central corneal scars (unpublished data).

Thus, the combination of OCT cornea thickness mapping and guided laser corneal surgery overcomes the limitations of prior art methods.

In a third aspect, the present invention also provides a method for performing femtosecond laser anterior keratoplasty, comprising the steps of: (a) obtaining a tomograph of the cornea of a subject with optical coherence tomography; (b) converting the optical coherence tomograph into a map of corneal thickness; (c) designing a laser dissection treatment plan base on the corneal thickness map; (d) performing intrastromal dissection according to the treatment plan using a femtosecond laser; (e) removing dissected anterior corneal tissues to leave a recipient bed; and (f) replacing the removed tissues with a disk of donated corneal tissue.

Anterior keratoplasty, also known as lamellar keratoplasty, is the medical procedure that involves replacement of the patient's diseased anterior corneal stroma and Bowman's membrane with donor material. Host endothelium, Descemet's membrane, and a part of the deep stroma are preserved. The donor corneal disc becomes repopulated with host fibroblasts, and the recipient epithelium usually covers the anterior corneal surface. This procedure is technically challenging. Methods of the present invention takes advantage of the precision and automation afforded by the high-precision systems of the present invention.

In particular, the present invention provides a novel approach to the design of the shapes of donor corneal shape to be excised and the recipient corneal bed to be formed. Example 4 provides further illustration of this aspect of the present invention.

In a fourth aspect, the present invention further provides a computer configured such that it is capable of automating and controlling a corneal mapping device in concert with an ablative laser to perform the methods of the present invention. Design and configuration of computer systems are generally known in the art. The computer may be a general purpose computer such as a PC or a special purpose computer specifically designed for the system such as a custom system employing specialized image processing accelerating circuitry or any other suitable computer hardware commonly known in the art.

Software programs that implements the methods and protocols of the present invention may also be designed using software design tools commonly known in the art. Exemplary software implementation tools may include JAVA, C, Fortran, or MATLAB. Other software implementations commonly known in the art may also be used.

In a fifth aspect, the present invention also provides computer readable medium having encoded thereon computer software that implements the methods of the present invention.

To further illustrate the present invention, the following specific examples and exemplary embodiments are provided.

### EXAMPLES

### 1. High-Speed Optical Coherence Tomography Mapping of the Cornea

OCT Image Capture

1. High-Speed OCT Scanning.

An OCT system with a speed of at least 2 kHz axial scan repetition rate is needed to accurately map corneal thickness. A speed of at least 20 kHz is needed to accurately map the anterior corneal surface elevation. An even higher speed is preferred for the mapping of highly irregular corneas because even a small movement within the scan acquisition period can lead to misregistration of a fine surface irregularity.

2. Meridional Scanning Pattern.

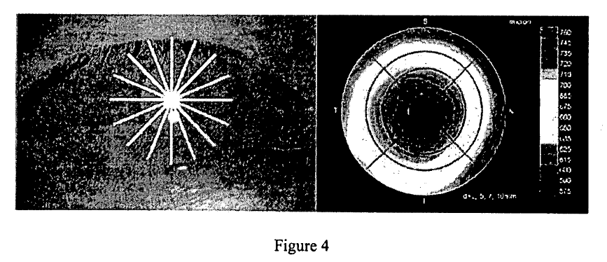

The strong specular reflection at the corneal vertex (the point on the corneal surface that is perpendicular to the visual fixation axis) is easily visible on the OCT image and serves as a reliable landmark. Radial lines centered on the vertex forms meridians. A meridional OCT scan has the special property that the OCT beam remains perpendicular to the corneal azimuth, and its incidence angle in the meridional plane can be measured from the image. This allows accurate dewarping to correct the image dimensions for the effect of refraction at the air-corneal interface. Thus, in the present exemplary embodiment, the best scan pattern consists of line scans across corneal meridians centered at the vertex. This spoke pattern as shown in FIG. 4 has the additional advantage of sampling the optically more important central region more densely than the periphery.

3. Telecentric Scan Geometry

The OCT beam preferably remain parallel to the optical axis of the instrument as it is scanned in the transverse dimension. This minimizes distortion in the resulting OCT image and makes dewarping easier.

4. Image Alignment

Pupil position information is preferrably captured at the same time that OCT scanning of the cornea is performed. While the vertex is the natural landmark for OCT imaging, the pupil (the opening within the iris diaphragm) is the preferred centration point for both excimer laser and femtosecond laser treatments. The OCT corneal map must be registered to the location of the pupil center. Thus the pupil position must be measured at the time of OCT corneal scanning. Preferably, the OCT scan has sufficient axial range to capture both the cornea and the iris in the image. This way, the inner edge of the iris (pupil border) can be exactly established in relation to the corneal map using OCT data alone. Alternatively, a coaxial en face camera image (preferably a digital camera) of the anterior eye can be taken during the OCT scan to visualize the position of the OCT scan pattern in relation to the pupil.

OCT Image Processing

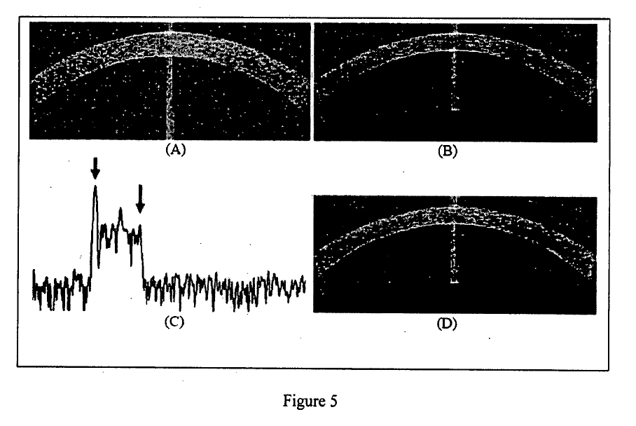

After corneal OCT images are captured, they are processed by a computer to map the position of corneal boundaries and the distance (thickness) between the boundaries (FIG. 5). The computer program performs the following functions:
1. Dewarp the OCT images to compensate for scan geometry and index transition at tissue boundaries (FIG. 5B).
2. Map air/tear, anterior stromal and posterior corneal boundary positions (FIGS. 5C, 5D). These are also called surface elevation maps or elevation topography.
3. Map the thickness of the tear+epithelium, stroma and total cornea along the surface-normal line or along the optical axis (FIG. 4 right).
4. Quality control software rejects OCT images with insufficient signal, shadowing and excessive motion. Motion is be detected by comparing vertex position between meridional scans within a mapping pattern and correlation between repeat elevation maps.
5. Measure corneal vertex and pupil center positions.

### 2. Excimer Laser Phototherapeutic Keratectomy (PTK)

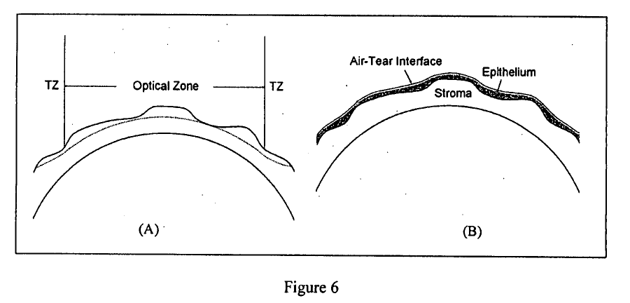

The goal of OCT-guided PTK is to remove tissue from the anterior corneal stroma with a precise depth pattern to remove opacities and restore a smooth anterior surface (FIG. 6A).

In the present invention, PTK laser ablation pattern can be designed from either corneal thickness or surface elevation map. If the posterior corneal surface is a good optical surface, then the thickness map is a better choice. Because the anterior and poster corneal surfaces move together with axial eye motion, thickness measurement is less susceptible to motion error. However, if the posterior surface is distorted (penetrating corneal scar or keratoconus), then the anterior elevation topography should be used.

If an OCT thickness map is used to plan the ablation, the ablation depth map is calculated to leave a constant thickness within the optical zone (FIG. 6A). A spherocylindrical ablation pattern could be added to the ablation pattern to produce a desired correction of refractive errors such as nearsightedness (myopia), farsightedness (hyperopia) and astigmatism. These patterns to correction refractive error are commonly used in photorefractive keratectomy (PRK) or laser in-situ keratomileusis (LASIK) and are well known.

If an OCT surface elevation map is used to plan the ablation, the ablation depth map is calculated to remove deviation of the elevation from a smooth spherical or parabolic target surface. The target surface is set at a minimal depth below the elevation map to minimize ablation.

It is not desirable to have a sudden step transition at the edge of the optical zone (FIG. 6A). Any discontinuity on the corneal surface provokes a severe healing (scarring) response and destabilizes the tear film. Therefore a transition zone is needed to gradually taper the ablation. The transition zone allows the ablation to transition from full correction within the optical zone to no ablation outside the transition zone.

Several methods are available for designing the transition zone ablation depth. A radial spline function is preferably used to match the depth and slope at the inner and outer radii of the transition zone.

Traditionally, PTK and PRK procedures are performed by first removing the epithelium with scraping, brushing or dilute alcohol solution to expose the anterior stromal surface, because the epithelium will grow back later and its thickness adds additional variability to the ablation process. However, transepithelial PTK is performed in some cases to take advantage of the epithelium as a smoothing agent for the ablation process. For OCT-guided PTK, the methods of epithelial removal and the choices of mapping parameters combine to form 8 options numbered in Table 1, of which 6 are acceptable methods that are discussed individually below:

TABLE 1
Feasible methods of using OCT corneal
maps to guide excimer laser PTK.

| | Method of deriving ablation depth pattern | | | |
|---|---|---|---|---|
| | Thickness-based OCT maps | | Elevation-based OCT maps | |
| | Total | | Air-tear | Anterior |
| Method of | corneal | Stromal | interface | stromal |
| epithelial | thickness | thickness | elevation | surface |
| removal | map | map | map | elevation map |
| Non-laser | 1 | 2 | 3 | 4 |
| Laser | 5 | No | 6 | No |

1. Laser ablation pattern based on total corneal thickness map (air-tear interface to posterior boundary), applied to the cornea after epithelial removal. The total thickness map is the most reliable OCT corneal map because the air-tear boundary is more sharply defined than the anterior stromal boundary. So the measurement is more reliable. However, epithelial removal exposes additional irregularity that is not captured on the total thickness map (FIG. 6B). Thus the ablation would not remove all irregularity. Due to its simplicity, this is the best option if the OCT resolution is not sufficient to reliably detect the anterior stromal surface boundary.

2. Laser ablation pattern based on stromal thickness map (anterior stromal surface to posterior boundary), applied to the cornea after epithelial removal. This would theoretically remove all corneal irregularity. However, it is more difficult to detect the anterior stromal boundary on OCT images and this option is only reliable on high-resolution OCT systems.

3. Surface-based variant of option 1.

4. Surface-based variant of option 2.

5. Laser ablation pattern based on total corneal thickness map (air-tear interface to posterior boundary), used to ablate both the epithelium and the underlying corneal stroma. This would theoretically remove all corneal irregularity as in option 2. Preferably the tear+epithelial thickness map is also used to plan the laser pattern because the ablation efficiency of the epithelium is slightly different from that of the stroma. Compared to option 2, this method is less sensitive to errors in detecting the anterior stromal surface because an error in the tear-epithelial thickness would only introduce a partial error in ablation depth proportional to the difference in ablation rate between the epithelium and stroma. Overall, this would be the best option if a high-resolution OCT system is available

6. Surface-based variant of option 5.

All of the surface-based methods require higher OCT scan speed to compensate for greater susceptibility to motion error. Thickness-based maps are less sensitive to axial motion than surface-based maps because the corneal layers move together. For example, in a system with 2 kHz axial scan repetition rate over an axial scan range of 3 mm in tissue, the time between axial scans is 0.5 msec. For a corneal thickness of 550 μm, the time for axial scan to cross the cornea is only 0.09 msec. An OCT image frame consisting 128 axial scan requires 64 msec to acquire. If the cornea moved at constant speed to traverse 10 microns axially over the 64 msec image acquisition time, the thickness measurement error due to motion is only 0.015 microns (in 0.09 msec). Therefore motion error is always smaller for thickness measurement compared to surface elevation measurement in OCT biometry.

### 3. OCT-Guided Transepithelial Photorefractive Keratectomy

Photorefractive keratectomy (PRK) treats ametropia by employing a 193 nm argon fluoride excimer laser to reshape the anterior corneal stroma by photoablation after removing the epithelium. The corneal epithelium is a highly active, self-renewing layer. A complete turnover occurs in approximately five to seven days. The epithelium is not uniformly thick therefore it could not be removed by a uniform ablation pattern. A uniform ablation pattern imposed on an unknown epithelial thickness would produce an unpredictable refractive effect.

In conventional PRK, the epithelium is removed by manual scraping, automated brush, automated microkeratome or alcohol. These procedures require separate epithelial removal step or equipment and may be associated with unnecessary epithelial removal or damage.

However, with OCT mapping of the epithelial thickness, the excimer laser can be programmed to remove the epithelium using a customized pattern. The advantages of transepithelial ablation are faster healing (no unnecessary epithelial removal or damage), fast and simple procedure (no separate epithelial removal step or equipment) and the ability of the epithelium to act as a masking agent to remove small scale irregularity.

Mapping Epithelial Thickness

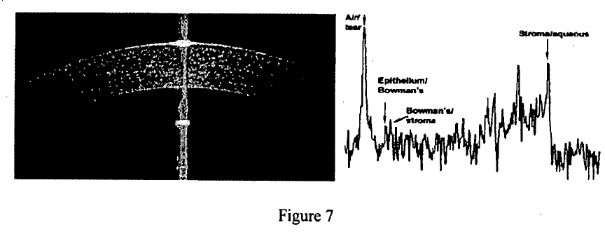

A very high-speed Fourier-domain optical coherence tomography (FD-OCT) cross-sectional image of a normal cornea was shown in FIG. 7 (left). The air-tear interface and the epithelium-Bowman's layer interface could be located by identifying the signal peak from the axial scan (FIG. 7, right). The OCT image was processed (“dewarped”) to remove the distortion due to refraction at the air-corneal interface and any deviation of the scan geometry from the ideal rectangular geometry. The epithelial thickness is the distance between the air-tear and epithelium-Bowman's layer interfaces. This “epithelial thickness” measurement includes both the epithelium and the tear film. The natural tear film is very thin and below the depth resolution of OCT in most eyes. For the purpose of guiding transepithelial ablation, it is not necessary to separate out the tear film.

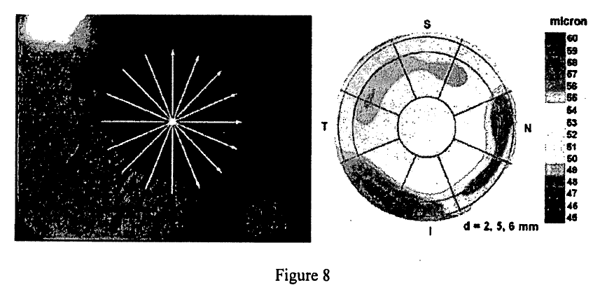

The epithelium thickness is measured along a line perpendicular to the corneal surface, or along a predefined absolute axis. For the purpose of PRK, the relevant thickness should be measured parallel to the optical axis of the laser system. Since the patient fixates on a coaxial target during the laser treatment, this axis is also parallel to the vertex normal (a line drawn perpendicular to the anterior corneal surface at the corneal vertex). By combining cross-sectional OCT scans on several meridians (FIG. 8, left), a map of the epithelium thickness is produced (FIG. 8, right). The epithelial thickness in the area between the meridional OCT scan are interpolated.

Epithelial Ablation Map

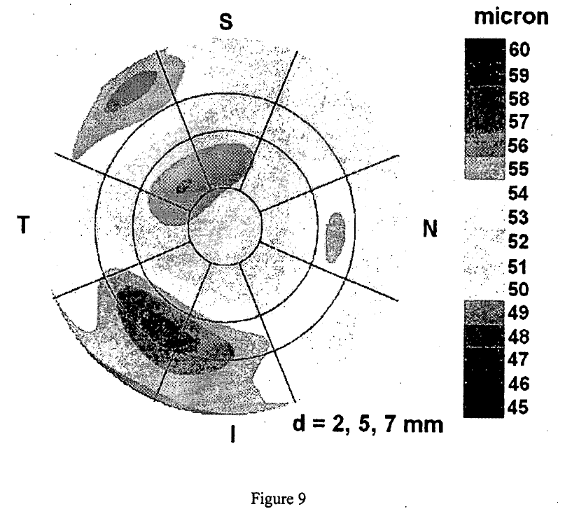

In OCT-guided transepithelial PRK, the OCT epithelial thickness map is used to devise a map of ablation depth that will remove the epithelium cleanly over the ablation zone. Because ablation in the optical zone produce direct refractive effect, it is important to have direct epithelial thickness measurement within the optical zone, which is usually 5.0 to 7.0 mm in diameter and centered on the pupil of the eye. The ablation zone in modern ablation design is often wider than the optical zone to incorporate a transition zone outside of the optical zone where the ablation depth gradually transitions to zero. The ablation in the transition zone has relatively little effect on visual outcome and therefore the epithelial thickness in the transition zone could be based on extrapolation if necessary. The design of the epithelial ablation map starts with the epithelial thickness map (FIG. 8 right). If the map is smaller than the ablation zone, then extrapolation is performed to extend the size of the map. Preferably the epithelial thickness extrapolated area is set to the epithelial thickness value at the edge of the directly measured area. The epithelial thickness map is then processed by low-pass spatial filtering. This step reduces the potential for the ablation to introduce high spatial frequency aberration due to errors in ablation pattern registration, eye movement during laser treatment or OCT measurement, and OCT measurement error. This also preserves the desirable effect of using the epithelium as masking agent so high-spatial-frequency irregularity of the corneal surface is smoothed out by the transepithelial ablation. The cut-off frequency of low-pass filtering is preferably lower than the epithelial smoothing action, which has been measured to be approximately 2 radian/mm (the inverse spatial constant is 0.5 mm/radian). The exemplary epithelial ablation depth map (FIG. 9) has been low-pass filtered with a 2 dimensional low-pass filter with a cut-off frequency of 1 radian/mm.

The epithelial ablation depth map is then used to generate the excimer laser pulse map, which will further take into account the laser spot size and fluence profile, spot placement, tissue removal rate and the variation in ablation efficiency due to variations in incidence angle on the cornea.

After removing the epithelium with the excimer laser, the surgeon can continue the laser ablation on corneal stroma. The stromal ablation pattern can be designed in the same way as currently practiced for photorefractive keratectomy (PRK) or laser in-situ keratomileusis (LASIK). The ablation pattern can be based on manifest refraction, topography or wavefront measurements. Alternatively, the laser ablation pattern could be based on an OCT pachymetry map or OCT topography map to treat an irregular cornea. Surface laser ablation of the cornea to remove irregularity is called phototherapeutic keratectomy (PTK). Since removal of irregularity and refractive correction can both be achieved in the same laser treatment session, the distinction between PRK and PTK can be blurred. When we describe transepithelial PRK we also include the possibility of a PTK (therapeutic) component.

Other imaging techniques, such as the ultrahigh frequency ultrasound imaging, could also map the epithelium thickness. Any of these epithelium measurement methods could be used in conjunction with present invention, although the OCT is preferred.

Transepithelial PRK can be used to remove small spatial scale (high spatial-frequency) irregularity from the cornea and thereby improve the quality of vision. This situation arises on corneas with previous refractive surgery, injury, infection, or intrinsic disease (epithelial basement membrane dystrophy, keratoconus). Transepithelial PRK also minimizes the area of epithelial removal and damage, thereby reducing postoperative discomfort and speeding recovery. This advantage is manifest even for a completely normal cornea. OCT-guided epithelial ablation is better than ablation with a flat beam (uniform ablation depth) because it reduces un-intended refractive shift and aberration due to non-uniform epithelial thickness. This improves the predictability of refractive outcome and improves quality of vision.

### 4. Femtosecond Laser Anterior (Lamellar) Keratoplasty (FLAK)

In keratoconus and other ectatic diseases, anterior lamellar keratoplasty is performed to restore the mechanical strength and stability of the cornea by replacing diseased tissue with a healthy lamellar transplant. The problem with anterior lamellar keratoplasty is that manual lamellar dissection leaves a rough interface. Deeper dissection to bare Descemet's membrane would provide a smooth surface, but risks perforation. The mechanical microkeratome can cut a smoother surface, but the diameter and depth of the cut is not precisely predictable, risking poor matching between donor and recipient tissue in some cases. The femtosecond laser can produce precise cuts, but current dissection program cuts at a constant distance from the anterior surface, leaving an irregular bed that does not match the more uniform donor tissue. A further limitation of the femtosecond laser is that deep (>200 micron from the anterior surface) cuts tend to leave a rough corrugated surface due to striae formation in the posterior stroma when the cornea is applanated. This example demonstrates how the present invention may be beneficially employed to overcome the limitations of the femtosecond laser to optimize anterior lamellar keratoplasty in keratoconus.

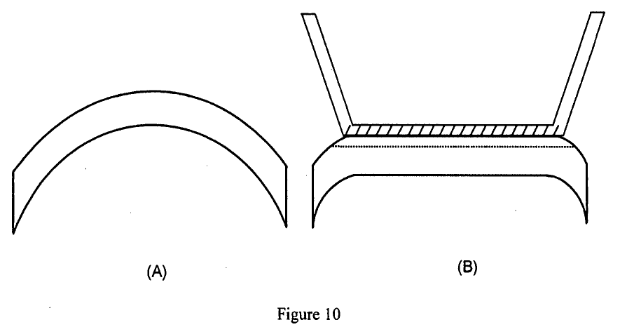

Referring to FIG. 10, the general procedure of this example is as follows: A contact plate is first applied to the cornea to applanate (flatten) the cornea. A femtosecond laser is then applied through the contact plate. The OCT corneal thickness map of the present invention is then used to guide the femtosecond dissection so it leaves a recipient bed of uniform thickness. Because the cornea is applanated by the contact plate, the design of ablation profile may be simplified by assuming a flat anterior surface for the cornea. To minimize unwanted sharp bends on the anterior and posterior corneal surface, a tapered edge is design to match the donor and recipient cornea.

Donor Cornea Preparation

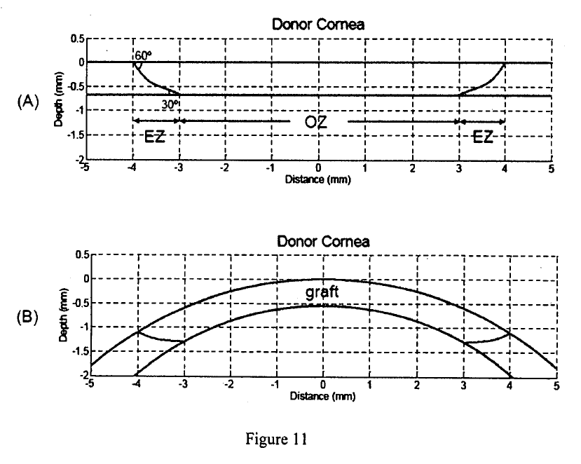

Referring to FIG. 11A, the donor cornea is left intact within the optical zone (OZ) and the Descemet's membrane is peeled off to leave a smooth posterior surface. The edge zone (EZ) will be cut with the femtosecond laser to create smooth tapered shape. The cut intercepts the anterior corneal surface at a 60-degree angle, or another angle preferably between about 45 and 90 degrees. The laser cut intercepts the posterior corneal surface at a 30 degree angle, or another angle preferably between 10 and 45 degrees. The femtosecond laser dissection can continue for a short distance beyond the expected full depth at the 30-degree trajectory to make sure that the laser cut is through the full thickness even in cases where the cornea is slightly thicker than expected.

A gradual transition in slope is used to connect the anterior and posterior cut edge. This curved slicing is preferably designed using a 3^{rd} order polynomial to meet the boundary conditions:
z=a₁x³+a₂x²+a₃x+1
where z is the vertical axis and x is the horizontal axis in FIG. 11B.

For instance, if the OZ radius is 3 mm, graft radius is 4 mm, the corneal thickness at the EZ (annular 3 mm-4 mm surrounding OZ, FIG. 11A) is assumed to be 670 micron (see reference 9, the relevant portion of which is incorporated by reference), the fitted polynomials for the boundary shape of the EZ are:
z=−0.9694x³−9.6014x²−32.0117x−36.4666 (left segment)
z=0.9694x³−9.6014x²+32.0117x−36.4666 (right segment)
After the applanation is released, the donor cornea restores to its normal shape (FIG. 11B).

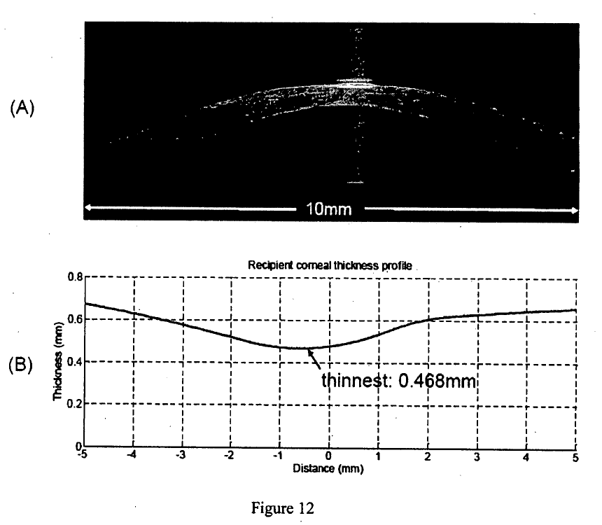

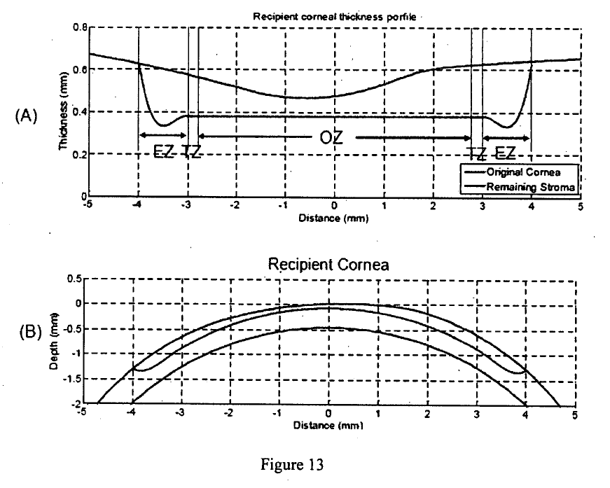

FIG. 12A shows an exemplary recipient cornea sectional image, FIG. 12B shows the corresponding corneal thickness profile. It can be seen here that the recipient cornea is thinner at the center than at the edges. FIG. 13A shows an exemplary design for the edge incision contour. The curvature at either edge (the section bounded within the EZ region) follows a polynomial curve shape. The cornea is shown here in an applanated state. FIG. 13B shows the corresponding cornea when restored to its unapplanted state.

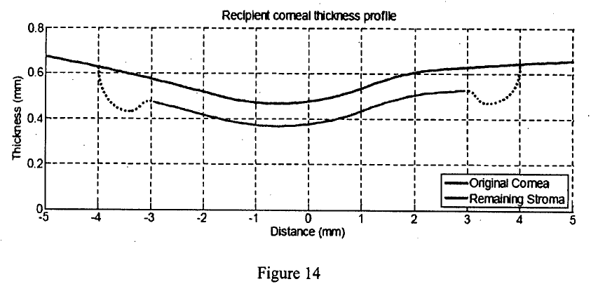

FIG. 14 shows another incision design that has a constant thickness throughout the OZ region.

The above exemplary designs are merely for illustrated purposes only. A person skilled in the relevant art will readily recognize that other configurations are also possible.

Preferably the design of the donor cornea dissection depth is performed after the donor corneal thickness map is measured by OCT at the eye bank. The laser dissection profile is customized for each meridian according to the OCT thickness profile. Alternatively, the donor cornea could be cut using a generic program designed for the average cornea. This would still work well because the dissection profile continues at 30 degrees angle to accommodate thicker corneas. The shape of the edge dissection does not vary much within the range of normal corneal thickness.

Recipient Cornea Preparation

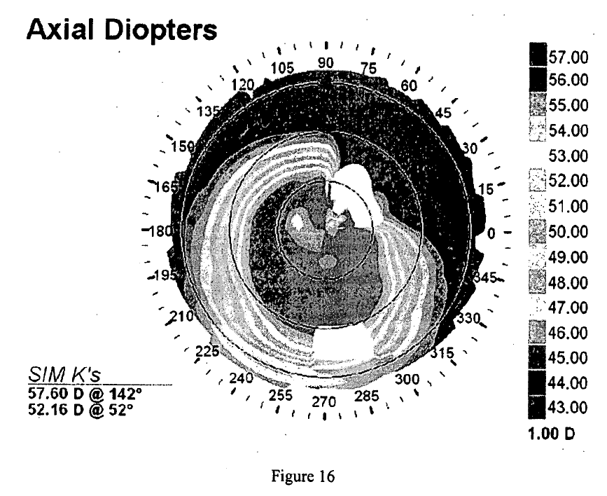

FIG. 16 shows the topography map of a keratoconic eye. FIG. 12A-B show its OCT image and pachymetry profile along the horizontal meridian.

The OZ of recipient cornea is slightly smaller than that of the donor cornea. A small annular region at the perimeter of the OZ is used as transition zone (TZ) to ensure a smooth transition from OZ to EZ. Within the OZ, the dissection depth is set to the OCT pachymetry map minus a fixed distance from the endothelium within the OZ. This will leave a bed of constant thickness in the OZ (FIG. 13A). The depth of the cut within the OZ is preferably between 80 microns (to ensure a level below the Bowman's layer) and 200 microns (to ensure a smooth cut). The minimum depth is the stricter limit if both cannot be fulfilled. In our keratoconus example (FIG. 12), the corneal thickness was 468 microns and 620 microns within the OZ (5.5 mm diameter). The bed thickness is therefore set at 388 microns (468-80). The laser dissection depth varies between 80 and 232 microns. The ablation profiles with applanation on (FIG. 6A) and after applanation is released (in FIG. 13B) are shown.

A simpler method for designing recipient OZ is to set a uniform depth to the anterior surface. The depth is preferably between 80 and 200 microns. In our example (FIG. 14) this was set at 100 microns. This will leave a recipient bed (FIG. 14) of variable thickness within the OZ. But because the donor cornea is much thicker, the irregularity will be suppressed after the donor cornea is sutured on. The advantage of this method is that it does not require a thickness map of the recipient cornea to be measured by OCT or other methods, and centration error during the laser dissection would be less critical.

To match the graft at the peripheral, the recipient EZ is designed so that it matches the donor cornea by three elements: (1) the size (equal outer diameter), (2) the angle at the outer edge, (3) the length of the cut surface traversed by the laser cut along the radial dimension. Following the above example of donor cornea, the EZ of the recipient cornea is from 3 mm to 4 mm, the same as the donor cornea. The outer edge of the EZ intercepts the anterior cornea surface at the same angle as the donor cornea (60 degrees). The inner edge of the EZ connects to the TZ by a smooth transition. In order to match the curve length of the EZ boundaries of the donor and recipient, a 4th order polynomial is used to design the EZ boundary of the recipient cornea. The fitted polynomials for the boundary shape of the EZ are:
z=−0.0001x⁴−0.6313x³−6.0815x²−18.9158x−19.8722 (left segment)
z=−0.0001x⁴+0.6313x³−6.0815x²−+8.9158x−19.8722 (right segment)

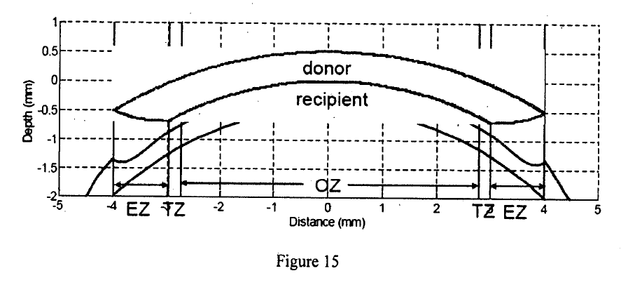

FIG. 15 shows the donor cornea on top of the recipient cornea. Because of the matching diameter, outer edge angle and interface length, there would be no abrupt curvature, slope or elevation change on either the anterior or the posterior corneal surface after the donor cornea is sutured in.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

1. Huang D, Swanson E A, Lin C P, et al. Optical coherence tomography. Science 1991; 254(5035):1178-81.
2. Huang D, Li Y, Radhakrishnan S, Chalita M R. Corneal and anterior segment optical coherence tomography. In: Schuman J S, Puliafito C A, Fujimoto J G, eds. Optical Coherence Tomography of Ocular Diseases, 2nd ed: SLACK, 2004.
3. Radhakrishnan S, Goldsmith J, Huang D, et al. Comparison of optical coherence tomography and ultrasound biomicroscopy for detection of narrow anterior chamber angles. Arch Opthalmol 2005; 123(8):1053-9.
4. Radhakrishnan S, Huang D, Smith S D. Optical coherence tomography imaging of the anterior chamber angle. Opthalmol Clin North Am 2005; 18(3):375-81.
5. Goldsmith J A, Li Y, Chalita M R, et al. Anterior chamber width measurement by high-speed optical coherence tomography. Opthalmology 2005; 112(2):238-44.
6. Huang D, Li Y, Radhakrishnan S. Optical coherence tomography of the anterior segment of the eye. Opthalmol Clin North Am 2004; 17(1):1-6
7. Ignacio T S, Nguyen T B, Chuck R S, et al. Top hat wound configuration for penetrating keratoplasty using the femtosecond laser: a laboratory model. Cornea 2006; 25(3):336-40.
8. Soong H K, Mian S, Abbasi O, Juhasz T. Femtosecond laser-assisted posterior lamellar keratoplasty: initial studies of surgical technique in eye bank eyes. Opthalmology 2005; 112(1):44-9.
9. Rufer F, Schroder A, Bader C, Erb C. Age-related changes in central and peripheral corneal thickness: determination of normal values with the Orbscan II topography system. Cornea 2007; 26(1):1-5.

What is claimed is:

1. A system for performing corneal surgery, comprising:
an optical coherence tomography device for mapping a cornea tomograph to a predetermined precision; and
an ablative laser linked to the optical coherence tomography device,
wherein actions of the ablative laser are guided by a treatment plan based on the cornea tomograph obtained by the corneal mapping device.

2. The system of claim 1, wherein the corneal mapping device is a Fourier-domain optical coherence tomography device.

3. The system of claim 1, wherein the optical coherence tomography device is capable of performing axial scans at a speed of at least 2 kHz.

4. The system of claim 1, wherein the optical coherence tomography device is capable of performing axial scans at a speed of at least 20 kHz.

5. The system of claim 1, wherein the ablative laser is an excimer laser.

6. The system of claim 1, wherein the ablative laser is a femtosecond pulsed laser.

7. The system of claim 1 further comprising a computer control unit configured to perform one or more programs wherein at least one of the programs is capable of controlling the actions of the ablative laser based on the treatment plan.

8. The system of claim 7, wherein at least one of the programs is capable of computing a cornea thickness map based on the tomograph obtained by the corneal mapping device and at least one of the programs is capable of generate a treatment plan for treating a refractive eye disorder based on the cornea thickness map.

9. The system of claim 8, wherein the treatment plan comprises an ablation pattern.

10. The system of claim 7, wherein at least one of the programs is capable of aligning the ablative laser with a target location on the eye in real-time by tracking movements of the eye.

11. The system of claim 1, wherein the optical coherence tomography device and the ablative laser form an integral unit encased in a housing such that both the optical coherence tomography and laser surgery can be performed at the same unit.

12. An eye surgery system, comprising:
a corneal thickness mapping device for generating a corneal thickness map; and
an ablative laser linked to the corneal thickness mapping device,
wherein actions of the ablative laser are guided by a treatment plan based on the corneal thickness map obtained by the corneal thickness mapping device.

13. The system of claim 12, wherein the ablative laser is an excimer laser.

14. The system of claim 12, wherein the ablative laser is a femtosecond excimer laser.

15. The system of claim 12, wherein the corneal thickness map measures the thickness of the cornea from front air-tear interface to the posterior boundary.

16. The system of claim 12, wherein the corneal thickness map measures the thickness of the cornea from the anterior stromal boundary, including Bowman's layer, to the posterior boundary.

17. The system of claim 12, wherein the ablative laser and the corneal thickness mapping device form an integrated unit encased in the same housing such that both the thickness map measurement and the ablative laser surgery can be delivered from the same unit.

18. An eye surgery system, comprising:
a optical coherence tomography device for mapping the corneal thickness of a subject; and
an ablative laser linked to the optical coherence tomography device,
wherein actions of the ablative laser are guided by a treatment plan based on the corneal thickness map derived from the optical coherence tomography measurements.

19. The system of claim 18, wherein the optical coherence tomography device is a Fourier-domain optical coherence tomography device.

20. The system of claim 18, wherein the optical coherence tomography has a precision of at least 2 micron.

21. The system of claim 18, wherein the ablative laser is an excimer laser.

22. The system of claim 18, wherein the ablative laser is a femtosecond excimer laser.

23. The system of claim 18, wherein the corneal thickness map is measured from the front air-tear interface to the posterior boundary.

24. The system of claim 18, wherein the corneal thickness map is measured from the anterior stromal boundary, including Bowman's layer, to the posterior boundary.

25. The system of claim 18, wherein the optical coherence tomography device and the ablative laser form an integral unit such that both the optical coherence tomography measurement and the laser surgery can be performed by the same unit.

26. A method for performing transepithelial photorefractive keratectomy, comprising:
obtaining a epithelial thickness map of a subject;
determining an ablation map based on the epithelial thickness map; and
removing epithelium tissue of the cornea with an excimer laser according to the ablation map.

27. The method of claim 26, wherein the epithelial thickness map is measure as the distance between the air-tear and epithelium-Bowman's layer interfaces.

28. The method of claim 26, wherein the epithelium thickness is measured along a line perpendicular to the corneal surface, or along a predefined absolute axis.

29. The method of claim 26, wherein measurement of epithelial thickness includes the area from about 5.0 to about 7.0 mm in diameter centered on the pupil of the eye.

30. The method of claim 26, further comprising processing the epithelial thickness map by a low-pass spatial filter, whereby the removal of epithelial tissue results in a smoothing of the cornea.

31. The method of claim 30, wherein the low-pass spatial filter has a cut-off frequency of 2 radian/mm or lower.

32. A method for performing laser phototherapeutic keratectomy, comprising the steps of:
a. obtaining a tomograph of the cornea;
b. generating a map of the cornea based on the tomograph, wherein the map comprise information of corneal thickness or anterior elevation of the cornea at a precision of at least 2 microns;
c. computing a treatment plan based on the cornea thickness map, wherein the treatment plan comprises ablation patterns to be performed by a laser; and
d. ablating the cornea with a ablative laser according to the ablation pattern of the treatment plan.

33. The method of claim 32, wherein the step of obtaining a tomograph further comprises capturing the location of the iris and the ablating step further comprises aligning the ablation pattern with the eye using the location of the iris as a reference.

34. The method of claim 32, wherein the ablation pattern is based on a cornea thickness map.

35. The method of claim 32, wherein the tomograph of the cornea is obtained at an axial scanning speed of at least 2 kHz.

36. The method of claim 32, wherein the corneal thickness is measured from the front air-tear interface to the posterior boundary.

37. The method of claim 32, wherein the corneal thickness is measured from the anterior stromal boundary, including Bowman's layer, to the posterior boundary.

38. The method of claim 32, wherein the ablative laser is a femtosecond laser.

39. A method for performing femtosecond laser anterior keratoplasty, comprising the steps of:
(1) obtaining a tomograph of the cornea of a subject with optical coherence tomography;
(2) converting the optical coherence tomograph into a map of corneal thickness;
(3) designing a laser dissection treatment plan base on the corneal thickness map;
(4) performing intrastromal dissection according to the treatment plan using a femtosecond laser;
(5) removing dissected anterior corneal tissues to leave a recipient bed; and
(6) replacing the removed tissues with a disk of donated corneal tissue.

40. The method of claim 39, wherein the laser dissection treatment plan is designed such that the corneal bed of the subject will have uniform residual thickness within a central optical zone.

41. The method of claim 40, wherein the depth of the cornea bed is constant at its outer edge, and of blended depth in a transition zone between the optical zone and the outer edge of the cornea bed.

42. A method for performing laser anterior keratoplasty, comprising:
preparing a donor cornea by dissecting with an ablative laser a portion of the anterior cornea which defines a donor disk having a tapered edge around the disk, wherein the dissection is guided by a treatment plan based on a corneal thickness map of the donor;
preparing a recipient cornea by dissecting with an ablative laser a portion of the anterior cornea to form a recipient bed having a substantially complementary edge shape to the donor disk, wherein the dissection is guided by a treatment plan based on a corneal thickness map of the recipient; and
applying the donor disk to the recipient bed to complete drafting of the donor material,
wherein the edge of the donor disk and the edge of the recipient bed are designed such that they form complementary curves.

43. The method of claim 42, wherein the ablative laser is a femtosecond excimer laser.

44. The method of claim 42, wherein the edge curve of the donor disk has a vertical cross-section that can be described by a 3^{rd} degree polynomial.

45. The method of claim 42, wherein preparation of the recipient bed is not guided by a corneal thickness map but dissected at a constant depth between 80 and 200 microns.

46. The method of claim 42, wherein the ablative laser is a femtosecond excimer laser.

47. A computer configured such that it is capable of controlling an ablative laser and a corneal thickness map device to performing the method of claim 26, 32, 39, or 42.

48. A computer readable medium having encoded thereon computer instructions for performing the method according to claim 26, 32, 39, or 42.
